(19) United States
(12) Patent Application Publication
(10) Pub. No.: US 2015/0374755 A1
(43) Pub. Date: Dec. 31, 2015

(54) Treated Cells and Therapeutic Uses

(71) Applicant: NHS BLOOD & TRANSPLANT, Hertfordshire (GB)

(72) Inventors: John Girdlestone, London (GB); Cristina Navarrete, London (GB)

(21) Appl. No.: 14/772,714

(22) PCT Filed: Mar. 6, 2014
(86) PCT No.: PCT/GB2014/050658

§ 371 Date: Sep. 3, 2015

(30) Foreign Application Priority Data
Mar. 6, 2013 (GB) 1304038.1

(51) Int. Cl. A61K 35/28; C12N 5/0775

(52) U.S. Cl. CPC A61K 35/28; C12N 5/0662; C12N 2501/04

(57) ABSTRACT

This invention relates to the use of mesenchymal stem cells in therapy. In particular it relates to a method of preparing the cells with enhanced immunosuppressive property and for use in the treatment of diseases of the immune system.

The present invention is in the field of mesenchymal stem cells (MSCs) and their use in therapy and research. In particular, the invention relates to treating or modifying the MSCs for suppressing unwanted immune reactions, such as graft—versus-host disease (GvHD) following stem cell transplantation, and rejection of transplanted organs, or treating autoimmune diseases.

Following an organ transplantation, the body will nearly always reject the new organ due to differences in human leukocyte antigens between the donor and recipient. As a result, the immune system detects the new tissue as “foreign”, and attempts to remove it by attacking it with recipient leukocytes, resulting in the death of the tissue. Conversely, with haematopoietic stem cell (HSC) transplants, the introduced immune system attacks the cells of the recipient, resulting in life-threatening GvHD.

Immunosuppressants are routinely applied as a countermeasure and are the main method of deliberately induced immunosuppression. In optimal circumstances, immunosuppressive drugs are targeted only at any hyperactive component of the immune system, and in ideal circumstances would not cause any significant immunodeficiency. However, all immunosuppressive drugs have the potential to cause immunodeficiency resulting in increased susceptibility to opportunistic infections and cancer.

Cortisone was the first immunosuppressant identified, but its wide range of side-effects limited its use. Azathioprine and cyclosporine followed, which allowed kidney transplantation to less well-matched donor-recipient pairs as well as liver transplantation, lung transplantation, pancreas transplantation, and heart transplantation. Although small molecules are in wide use they are not without their drawbacks. Achieving target doses is complicated by the lipophilic nature of many of the drugs, whereby the majority are sequestered in non-target cells such as red blood cells. Systemic administration also results in undesirable side effects.

GvHD is a leading cause of mortality associated with haematopoietic stem cell transplants. Severe GvHD can cause blistering of the skin, intestinal haemorrhage and liver failure. The condition is extremely painful with a death rate of up to 80%. At present, the first-line standard therapies for GvHD are steroids. Given that the success rate of steroids is only 30% to 50%, the only other therapy, if steroids fail, is limited to immunosuppressive agents that are used off-label with little benefit and significant toxicities.

In view of the limitations and disadvantages of current therapies, attention is now turning to cell based therapies, in particular MSCs (Bernardo and Fibbe). Numerous studies have demonstrated that human MSCs avoid allorecognition, interfere with dendritic cell and T-cell function, and generate a local immunosuppressive microenvironment by secreting cytokines. MSCs are immunomodulatory, multipotent and fast proliferating. These unique capabilities mean they have the potential to be used for a wide range of treatments.

Currently, there are numerous clinical trials involving allogeneic-derived adult stem cells (Clinicaltrials.gov accessed 14-01-13), and preliminary results have led to the licensing in several jurisdictions (Canada, New Zealand) of MSC-based cell therapy for steroid-resistant GVHD in children. The majority of current clinical trials are designed to examine the ability of MSCs to ameliorate tissue damage caused by ischaemia or immune responses. For example, MSC are being tested for treatment of: Myocardial infarction, cerebral strokes, limb ischaemia, spinal cord injuries, burns, fistulas; immune disorders such as ulcerative colitis, Crohn's disease, multiple sclerosis, Type I Diabetes, and Lupus; degenerative diseases such as Parkinson's, ALS, and liver cirrhosis; and for immune complications of stem cell and solid organ transplantation such as steroid-resistant GvHD, HSC engraftment failure, solid organ rejection, organ failure, chronic allograft nephropathy and fibrosis.

Challenges to the allogeneic stem cell therapy approach primarily reside in the manufacture of the cellular product (see Bernardo and Fibbe). Large numbers of cells must be produced per lot to satisfy the larger dosing requirement which may exceed 10⁶ cells/kg/dose. Optimal doses remain to be determined and may well be higher, depending on the application. To achieve the required cell numbers from individual sources requires weeks to months of tissue culture, which is carried out in media containing xenogeneic factors (e.g. fetal calf serum) or material of human origin (e.g. platelet lysate) that must be screened for known pathogens. Although large scale expansion from a single donation is desirable in terms of generating cell batches that are logistically and economically feasible to test for release criteria, concerns over karyotypic instability and neoplastic transformation limit the number of passages that can be carried out.

Additionally, the cells must be efficiently expanded in culture while retaining their proper cell characterization profile and efficacy. The cellular product must be amenable to cryopreservation and subsequent revival in order for the “off the shelf” production model to be successful. The successful manufacture of these products must also rely on a stringent quality control policy to demonstrate lot-to-lot consistency and safety.

The present invention attempts to alleviate some of the above problems.

According to the present invention therefor there is provided isolated mesenchymal stem cells obtained from a source treated with one or more immunosuppressive agents to provide MSCs with enhanced immunosuppressive potency when compared to untreated MSCs prior to use in therapy.

The immunosuppressive agent may be selected from rapamycin (Sirolimus), Everolimus FK506 (Tacrolimus) or cyclosporin A.

Preferably, the MSCs may be treated with the immunosuppressive agent for less than 24 hours, for example the MSCs may be treated for less than 12, 6, 4, 2 hours or less than 1 hour.

The MSCs may be obtained from a human source and need not be from a human embryonic source. The MSCs may be obtained from a non-human mammal. The MSCs may be derived from umbilical cord, bone marrow, adipose tissue, umbilical cord blood, or placenta.

The MSCs may be allogeneic or autologous. MSCs may be sourced from other species, for use in members of the same species, or xenogeneically.

Preferably, the MSCs may be sourced from human bone marrow or umbilical cord.

The MSCs can be autologous or allogeneic to the host that is being administered the treatment with MSCs. The allogeneic MSCs can be obtained from a donor or a third party source.

A combination of MSCs according to the present invention and other therapeutic agents may be used in therapy.

As used herein, increased immunosuppressive potency means an enhanced immunosuppressive activity. Particularly it relates to an enhanced immunosuppressive activity compared to equivalent MSCs which have not been treated with an immunosuppressive agent as defined herein.

The increased immunosuppressive potency may be an enhanced ability to suppress T lymphocyte effector function. The T lymphocytes may be CD4+ and/or CD8+ T lymphocytes.

The suppression of effector function may be a reduction in T lymphocyte proliferation. This may be determined using methods known in the art. For example, MSCs may be co-cultured with T lymphocytes and the rate of T lymphocyte proliferation assayed by thymidine incorporation, CSFE staining or other methods well known in the art.

MSCs treated with an immunosuppressive agent as defined herein may reduce T lymphocyte proliferation by 2-, 4-, 10-, 50-, 100- or 1000-fold compared to equivalent untreated MSCs.

The suppression of effector function may be a reduction in effector cytokine production by T lymphocytes. For example, treated MSCs may reduce the level of one or more of the following illustrative, non-exhaustive list of cytokines: IL-1, IL-2, TNFα, GM-CSF or IFNγ.

According to another aspect of the invention there is provided a method of preparing MSCs with increased immunosuppressive potency comprising the steps of obtaining MSCs from a source, culturing in a media, treating with an immunosuppressive agent for a period and harvesting the cells.

The cells treated in the method of the invention may be any primary cell or a cell line. For example the cell may be, but is not restricted to, primary fibroblasts, fibroblast cell lines, endothelial cells or haematopoietic cells.

The cells may be MSCs, human umbilical vein endothelial cells (HUVEC), primary adult human dermal fibroblasts (HDF) or antigen presenting cells (APCs). APCs which may be treated in the method of the invention include monocytes, dendritic cells and B cells.

Standard tissue culture conditions, media and supplements may be used.

The immunosuppressive agent may be selected from rapamycin (Sirolimus), Everolimus, FK506 (Tacrolimus), or any other agents with immunosuppressive properties.

Other agents that do not suppress the functional activity of immune cells on their own, but which increases the immunosuppressive potency of MSCs may also be used either alone or in combination with immunosuppressive agents such as rapamycin.

Agents that can suppress the functional activity of immune cells on their own, but which act synergistically with MSCs to immunosuppress, may be used either alone or in combination with immunosuppressive agents such as rapamycin.

The culture media may be selected from DMEM, DMEM:F12 mixtures, or other standard basal media used for culture of fibroblastic cell types. Basal media are supplemented with 10% fetal calf serum, or serum-free growth factor alternatives, and standard antibiotics such as Penicillin, Streptomycin, or Gentamicin. Cells may be cultured under standard conditions of temperature (37-38° C.) and CO2 (5%).

The MSCs may be exposed to the immunosuppressive agents for 0 to 24 hours, preferably for 1 hour or less. The MSCs may be treated with the immunosuppressive agent for less than 24 hours, for example the MSCs may be treated for 12, 6, 4, 2 hours or less, or less than 1 hour.

The MSCs according to the present invention may be used in medicine or for human or veterinary applications.

The MSCs according to the invention may be used to suppress an adverse immune response in a subject such as GvHD following organ or stem cell transplantation, and rejection of transplanted organs. The MSCs may be used for treating autoimmune diseases or other conditions where suppression of the immune system is required.

In another aspect there is provided a pharmaceutical composition comprising mesenchymal stem cells according to the present invention in a therapeutically effective amount and a pharmaceutically acceptable carrier, diluent or excipient.

The pharmaceutical preparation may be administered to a recipient in need thereof in an amount effective to reduce or illuminate an adverse immune response caused by a donor transplant against the recipient or host.

The compositions may be in a form that is suitable for delivery to a patient such as, tablets, capsules, powders, granules, elixirs, lozenges, suppositories, syrups and liquid preparations including suspensions and solutions.

The term “pharmaceutical composition” in the context of this invention means a composition comprising an active agent and comprising additionally one or more pharmaceutically acceptable carriers or suspension medium. The composition may further contain ingredients selected from, for example, diluents, adjuvants, excipients, vehicles, preserving agents, fillers, disintegrating agents, wetting agents, emulsifying agents, suspending agents, sweetening agents, flavouring agents, perfuming agents, antibacterial agents, antifungal agents, lubricating agents and dispersing agents, depending on the nature of the mode of administration and dosage forms.

The pharmaceutical compositions of the invention may be administered orally in any orally acceptable dosage form, including, but not limited to tablets, dragees, powders, elixirs, and syrups, liquid preparations including suspensions, sprays, inhalants, tablets, lozenges, emulsions, solutions, cachets, granules and capsules. Such dosage forms are prepared according to techniques known in the art of pharmaceutical formulation. When in the form of sprays or inhalants the pharmaceutical compositions may be administered nasally. Suitable formulations for this purpose are known to those skilled in the art. The pharmaceutical compositions of the invention may be administered by injection or intravenously and may be in the form of a sterile liquid preparation for injection, including liposome preparations. The pharmaceutical compositions of the invention may also be in the form of suppositories for rectal administration. These are formulated so that the pharmaceutical composition is solid at room temperature and liquid at body temperature to allow release of the active compound.

The inventors have demonstrated for the first time that brief exposure of human MSCs to immunosuppressive agents, such as Rapamycin, markedly increased the immunosuppressive potency of the MSCs.

The advantage of having enhanced immunosuppressive MSCs is that far fewer cells will be required for effective therapy, thereby reducing side effects, improving traceability, reducing cost and reducing the demands on large scale manufacture.

MSCs with high immunosuppresive potency may allow for achievement of clinical end-points that are not reached with standard dosing regimens.

In another aspect, MSC may be replaced with any primary or cultured cell type or cellular preparation that is to be infused or transplanted into a recipient.

It is proposed that the principal mechanism responsible for ‘super-suppression’ described herein involves the absorption of Rapamycin (or Everolimus, Tacrolimus, etc.) by MSC. These drugs are hydrophobic and exhibit high partition coefficients, such that they rapidly transfer into cells from the medium or plasma (Yanez et al.). The treated cells then serve as a reservoir for the drug which is available for transfer to other cells, such as lymphocytes, when they are placed in proximity to the MSC. This transfer is a passive process governed by physical parameters such as diffusion along concentration gradients.

Rapamycin inhibits the function of a kinase complex (mammalian (or mechanistic) Target of Rapamycin 1 (mTORC1)) that serves as a critical sensor of the nutrient and energy status of a cell. TOR inhibition can block the proliferative capacity of many cell types and is being tested for anti-cancer treatments (Borders et al.), although lymphocytes appear to be particularly sensitive to Rapamycin and this effect is exploited for clinical immunosuppression. The differential responsiveness to lymphoid subsets to Rapamycin can promote an anti-inflammatory balance, since T regulatory cells are relatively less sensitive to the drug than T effector cells (Thompson et al.).

The invention will now be described in the following examples and drawings by way of illustration only, in which:

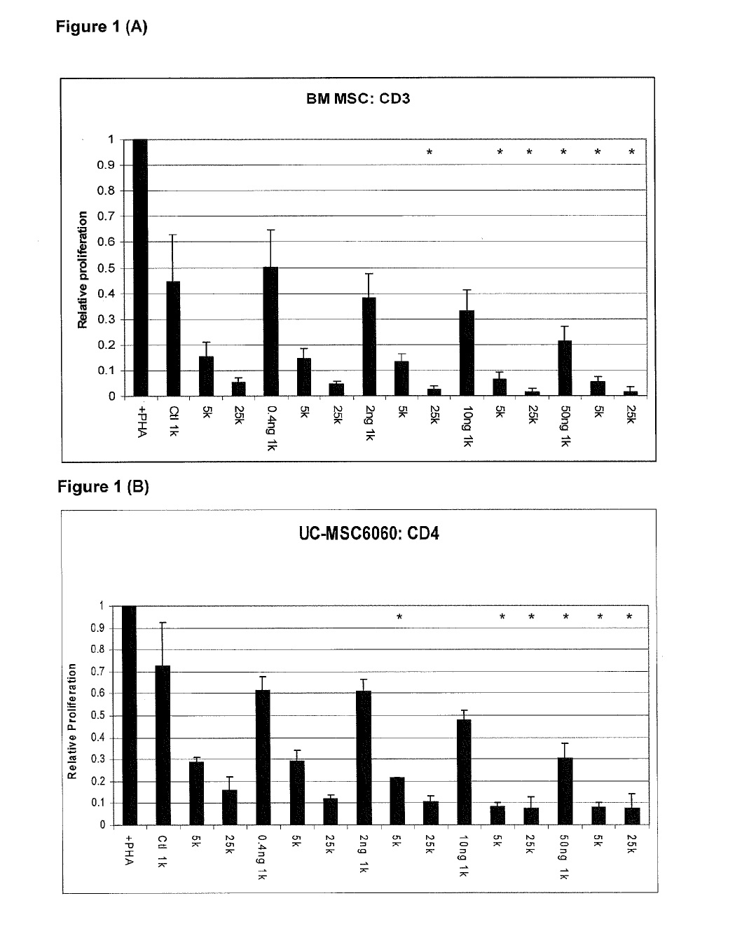

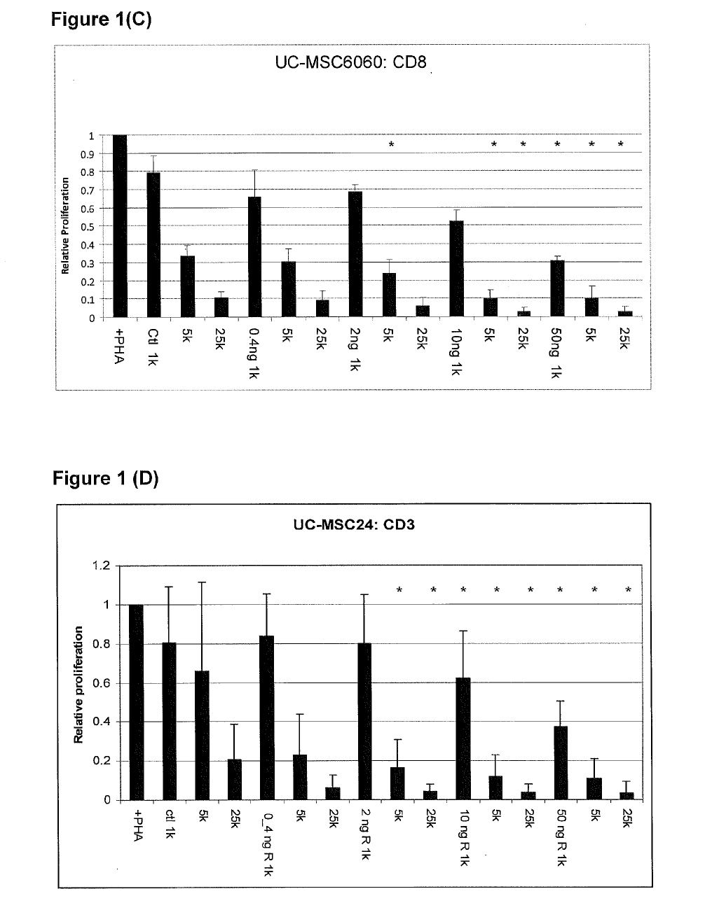

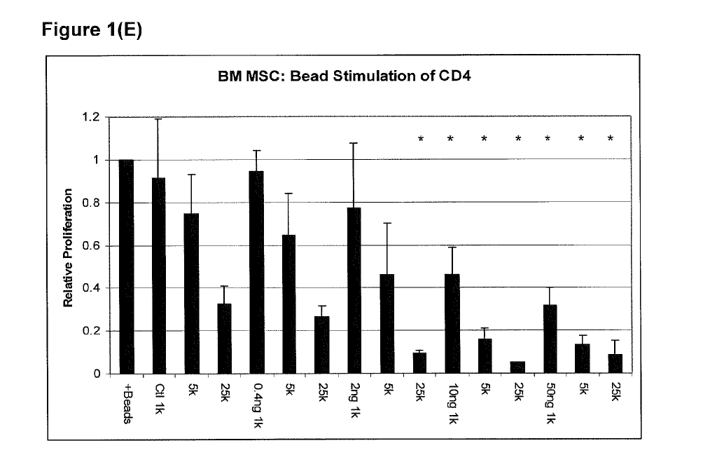

FIGS. 1A-E are graphs showing super-suppression of PHA- or activation bead-stimulated T Cell proliferation by Rapamycin-treated MSC from multiple sources,

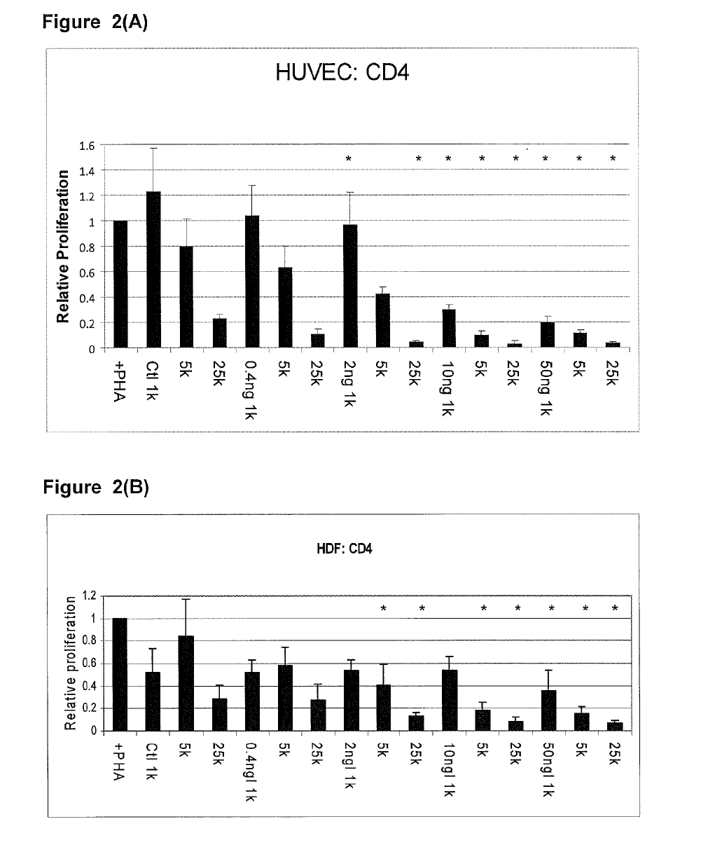

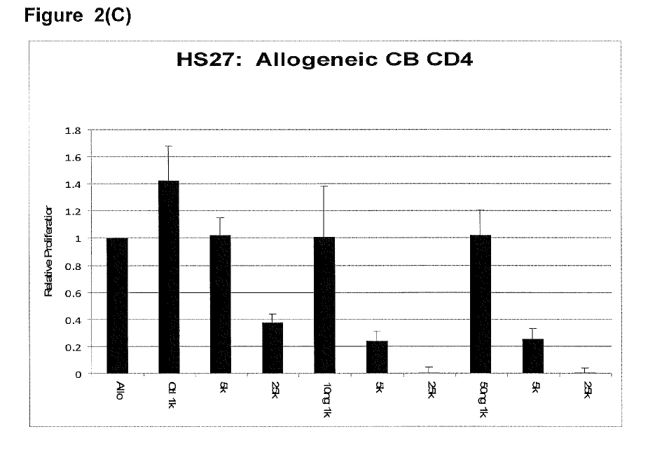

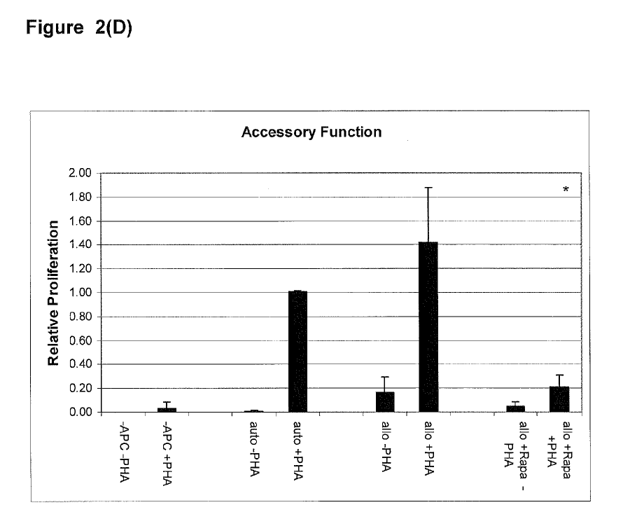

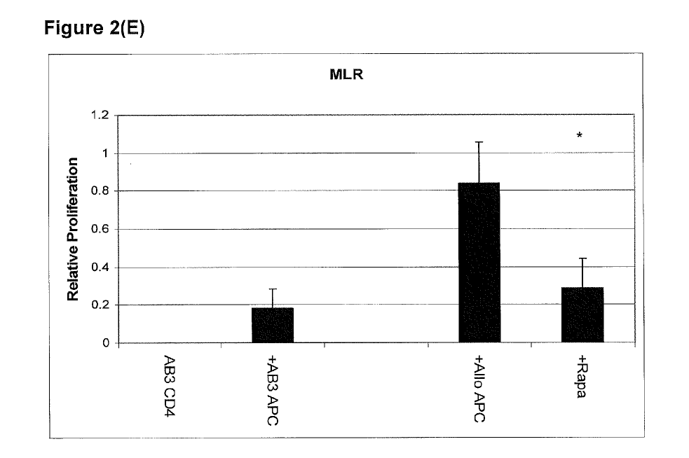

FIGS. 2A-E are graphs showing super-suppression of T Cell proliferation by other types of primary cells and cell lines with Rapamycin,

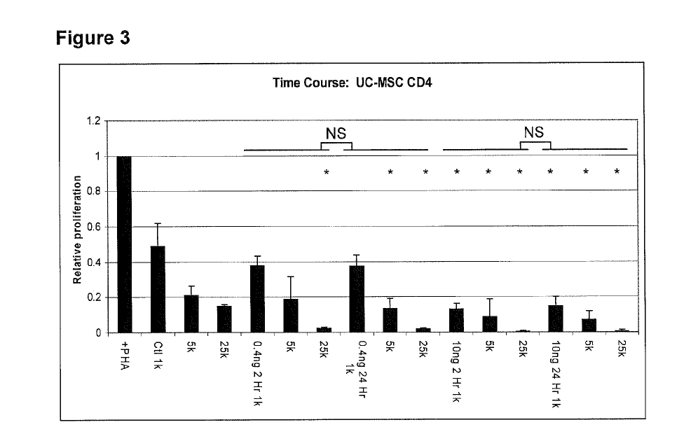

FIG. 3 is a graph showing that induction of super-suppression requires only short incubation times,

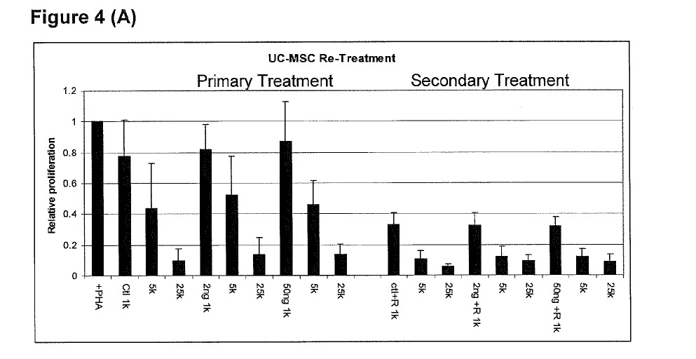

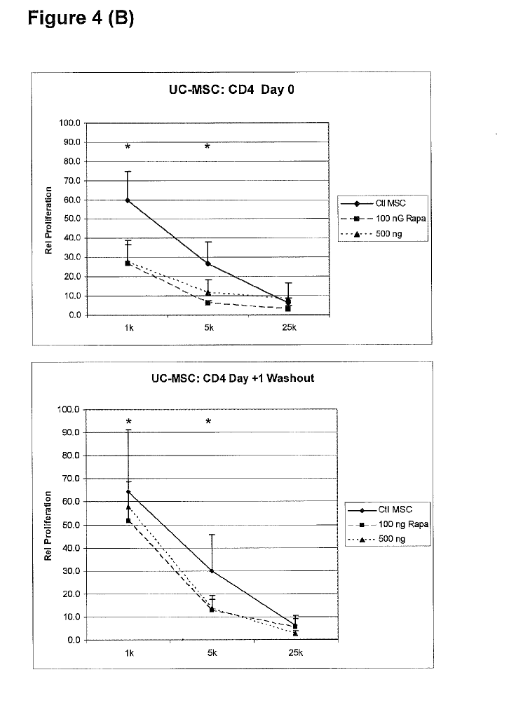

FIGS. 4A-B are graphs showing that Rapamycin does not induce a permanent increase in suppressive activity, or inhibit re-induction of super-suppression,

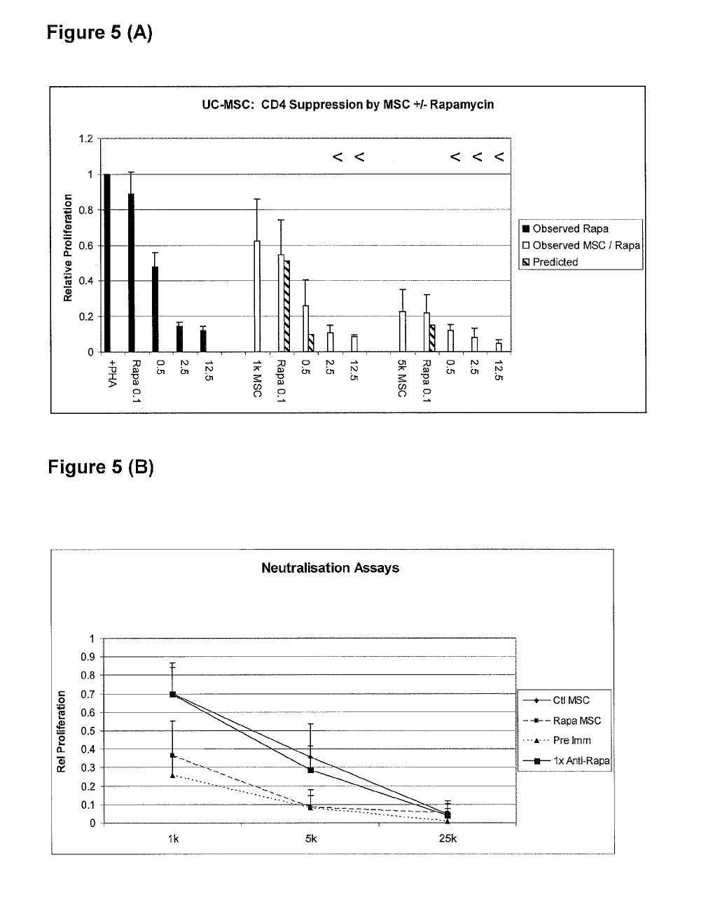

FIG. 5A-B are graphs showing that the suppressive effects of MSC and Rapamycin are additive, and that the super-suppressive effect is blocked by an anti-Rapamycin Ig.

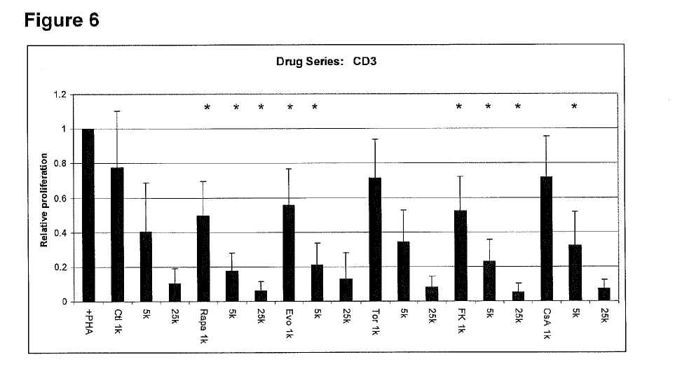

FIG. 6A is a graph showing that the suppressor effect is seen with Everolimus and Tacrolimus, but minimally with Cyclosporin or Torin1.

### EXAMPLES

### Example 1

Preparation of MSCs from multiple sources with enhanced immunosuppressive potency.

Both BM and UC-derived MSC can be made super-suppressive by pre-incubation with Rapamycin in a dose-dependent manner, such that they exhibit increased potency to inhibit induced proliferation of CD4 and CD8 T lymphocytes.

MSC derived from (FIG. 1A,E) bone marrow (BM) or (FIG. 1B-D) two independent umbilical cord (UC) preparations (WJ6060:FIG. 1B-C; WJ24-0: FIG. 1D) were incubated with Rapamycin overnight at concentrations from 0.4 to 50 ng/ml, or without drug (control (Ctl)). The MSC were washed, trypsinised, and plated in fresh medium without Rapamycin at 1, 5, and 25 k per well in 96 well plates. After 2-4 hours, human adult peripheral blood mononuclear cells (MNC) labelled with carboxyfluorescein succinimidyl ester (CFSE) were added, and the T lymphocytes therein were stimulated to proliferate with Phytohemagglutinin (PHA) (FIG. 1A-D) or anti-CD3, CD28 activation beads (FIG. 1E). Proliferation levels were determined after 3 days as described in the Methods section. For this and subsequent figures, values are presented as the averages from 3 or more independent MNC donors, with error bars representing Standard Deviation, and asterisks indicating a Student's t-Test with p<0.05 from comparison with equivalent cell numbers of Control (untreated) MSCs. The ‘+PHA’ value is the proliferation index obtained by stimulation in the absence of MSC, and is defined as 1 (see Methods).

### Example 2

Super-suppression of T cell proliferation by other types of primary cells and cell lines pre-incubated with Rapamycin.

The super-suppression of T Cell proliferation by other types of primary cells and cell lines which have less intrinsic suppressive activity than MSC is shown in FIG. 2. Rapamycin-treated (A) Human umbilical vein endothelial cells (HUVEC) and (B) primary adult human dermal fibroblasts (HDF) were tested for suppressive capacity as for FIG. 1. (C) HS27, a human fibroblastoid cell line, was pre-treated with 10 and 50 ng/ml Rapamycin for 3.5 hours and tested against cfse-labelled CD4+ lymphocytes that were isolated from Cord Blood (CB) and stimulated with allogeneic CB antigen-presenting cells. (D) Purified adult human CD4+ T lymphocytes were stimulated by PHA with or without autologous (auto) or allogeneic (allo) antigen presenting cells (APC), which were untreated or pre-incubated with 50 ng/ml

Rapamycin (+Rapa) for 1 hour then extensively washed. APC are required to provide an accessory function for induction of T cell proliferation by PHA. Proliferation was determined on day 4, a time point that is sufficient to detect PHA induction, but too brief for allogeneic stimulation. (E) Parallel cultures to those in (D) were assayed at day 6 to measure allogeneic stimulation of CD4 T cell proliferation (Mixed Lymphocyte Reaction (MLR)) in response to two different donors (values are averages of 3 different recipients challenged individually with 2 different stimulators).

It can be seen from FIG. 2A-C that not only MSCs but other cells treated with rapamycin exhibit increased suppression of T-cell proliferation. The observation that the induction of an immunosuppressive effect by Rapamycin pre-treatment is not limited to MSC, but is seen with primary cells (HDF, HUVEC) or fibroblastic cell lines (HS27) indicates, but does not preclude, that the drug rather than the cell is the primary ‘super-’ suppressive agent. Pre-treatment of an APC preparation (a mixture of monocytes, B cells and Dendritic cells) with Rapamycin significantly reduces the resultant T cell proliferation when the APC are provided as accessory cells (FIG. 2D), or as allo-antigenic stimuli (FIG. 2E).

### Example 3

Induction of Super-suppression requires only short incubation times.

Experimental conditions used were the same as in Example 1 for FIG. 1, with umbilical cord MSC (line WJ24-O) incubated with 0.4 or 10 ng/ml Rapamycin for 2 or 24 hours. There was no significant (NS) difference in the super-suppression between the two incubation periods (FIG. 3). The observation that brief exposures (˜1 hour or less (see also FIGS. 2D_E)) of MSC to the drugs are sufficient for maximal effect indicates a physical association, but does not preclude subsequent biological mechanisms of action on the MSC.

### Example 4

Rapamycin does not induce a permanent increase in suppressive activity, or inhibit re-induction of super-suppression.

Wash-out experiments and re-treatments were performed to determine if Rapamycin induces a permanent increase of the suppressive potency of MSC. Umbilical cord MSC (sub-line WJ24-O-2E) were incubated with 0, 2 or 50 ng/ml Rapamycin for 16 hours, then passaged 3 times over 13 days in drug-free medium. Parallel sets of cultures for each initial condition were then subjected to a secondary treatment with 50 ng/ml Rapamycin for 16 hours for suppression assays, prepared as in Example 1, FIG. 1. The MSC which were treated with Rapamycin at the beginning of the experiment (‘primary treatment’) and then cultured in drug-free medium for 2 weeks showed a degree of suppression that was no different to control MSC when tested at the end of the two week culture period (FIG. 4A). When re-treated with 50 ng/mL Rapamycin after two weeks (‘secondary treatment’), they showed the same elevated suppression as seen with MSC which had never been treated with drug (ctl+R).

The results of FIG. 4A demonstrate that the Rapamycin effect is transient, and a washout experiment was performed to determine the kinetics of decay (FIG. 4B). Umbilical cord MSC (WJ24-O) were incubated with 0, 100 or 500 ng/ml Rapamycin for 2 hours and one aliquot of cells for each condition was plated for T cell suppression assays as for FIG. 1 (FIG. 4B day 0). A second aliquot was re-plated into T25 flasks for an overnight culture in fresh medium without drug. At 24 hours after the initial treatment, these cultures were then prepared for T cell suppression assays as above (FIG. 4B Day +1). Approximately half of the suppressive effect due to the drug was lost after one day of culture in its absence, and no difference was seen between the 100 and 500 ng/ml doses.

### Example 5

The suppressive effects of MSC and Rapamycin are additive, and the super-suppressive effect is blocked by an anti-Rapamycin Ig.

To test for interactive effects of MSC and Rapamycin, MNC were stimulated with PHA in the presence of 0.1, 0.5. 2.5 or 12.5 ng/mL Rapamycin, with or without 1 k, 5 k and 25 k Cord MSC as for previous examples. The relative amounts of CD4 T cell proliferation were determined for each condition, and the levels of suppression mediated by Rapamycin, or MSC were added to obtain a Predicted level of suppression (FIG. 5A). Greater than 100% levels of predicted suppression are indicated by ‘<’. The observed levels of suppression mediated by combinations of

MSC and drug are similar to, if not less than, the predicted values indicating that there are no synergistic interactions.

Neutralisation experiments were carried out to determine if the super-suppressive effect was due to the action of Rapamycin released from pre-incubated MSC. Cord MSC were incubated with 50 ng/ml Rapamycin for 1.25 hours and plated along with control MSC as in Example 1 and FIG. 1. After 2-4 hours culture to allow MSC adhesion, sheep anti-Rapamycin Ig (anti-Rapa) was added at 0.4 uG/well (2 uG/mL), and parallel wells received pre-immune serum (Pre Imm). After a further 30′ incubation, cfse-labelled MNC +/−PHA were added, and 4 days later proliferation was measured (FIG. 5B). The pre-immune serum showed no effect in this assay, and further specificity controls demonstrated that the anti-Rapamycin Ig did not block the super-suppressive effect of FK-506-treated MSC (not shown).

The observation that a neutralising antibody directed against Rapamycin is sufficient to revert the immunosuppressive activity of treated MSC back down to the activity exerted by untreated MSC indicates that the increased immunosuppression is due primarily to the effect of Rapamycin (or an immunologically cross-reactive metabolite) that has been introduced into the culture by the pre-treated MSC and made available for action on the lymphocytes.

### Example 6

Suppressor effect seen with Everolimus and Tacrolimus, but minimally with Cyclosporin or Torin1

To determine if the super-suppressive effect of Rapamycin could be replicated with other immunosuppressive drugs, MSC were pre-incubated with the Rapamycin analogue Everolimus, Torin1 (an mTOR inhibitor), FK506 (Tacrolimus), and Cyclosporin A. All were used at 50 ng/ml according to the procedures used for Example 1. As shown in FIG. 6 the ‘rapalogue’ Everolimus, and Tacrolimus showed similar degrees of super-suppression to those seen for Rapamycin, but Cyclosporin A pre-treatment of MSC at 50 ng/ml or higher doses (not shown) did not result in substantial super-suppression. The observation of increased immunosuppression by MSC treated with Tacrolimus, which has a different mode of action to Rapamycin, indicates that the MSC-mediated effect is independent of the cellular target of the drug. Rapamycin's principle action is thought to be inhibition of the mTor complex 1, but no significant super-suppression was seen with Torin 1, which inhibits mTor through a different mechanism (Thoreen et al.). Therefore, the relevant parameters for super-suppression may involve the physico-chemical nature of the agent, specifically the ability of the drug to partition into the MSC when introduced into the culture medium. The lipophilic nature of drugs such as Rapamycin and Tacrolimus leads to their partition into cells rather than plasma (36:1 ratio, see Yanez et al.)

Methods

MSC and Fibroblasts.

Umbilical Cord MSC (UC-MSC) were generated as described (Girdlestone et al.) from fresh cord segments collected from full-term births by NHS Cord Blood Bank (NHS-CBB) staff (Colindale, UK) after obtaining informed ethical consent. UC-MSC was used up to passage 15 with no apparent loss of immunomodulatory potency. Bone Marrow (BM) MSC were generated by standard methods from frozen aliquots of mononuclear cells (MNC) purchased from DV Biologics (Costa Mesa, Calif., USA). Briefly, the MNC were thawed and plated in a tissue culture flask with standard growth medium: DMEM:F12 (Lonza, Basel, Switzerland) supplemented with Penicillin/Streptomycin (Sigma, Poole, UK) and 10% fetal calf serum (FCS) (Life Technology, Paisley, UK). Cells were cultured at 38° with 5% CO2 and passaged using trypsin. The MSC phenotype was assessed by flow cytometry for the presence and absence of surface markers CD73, CD90, CD45, CD34; Biolegend, London, UK) as described (Girdlestone et al.).

The HS27 human foreskin fibroblast cell line (ECACC, Porton Down, UK) and primary human dermal fibroblasts (TCS Cellworks, Buckingham, UK) were grown under the conditions used for MSC. HUVEC purchased from ECACC were expanded in endothelial cell growth medium (TCS Cellworks).

Cell Proliferation

Adult peripheral blood mononuclear cells (MNC) were obtained by centrifugation of apheresis cone contents over a Ficoll cushion. MNC were labelled with 1.25 uM carboxyfluorescein succinimidyl ester (CFSE) at room temperature for 15′ in order to monitor proliferation, and added to the MSC plates at 50k/well in the presence of 0.5 ug/ml phytohaemagglutinin (PHA), or CD3/CD28 activation beads (Miltenyi, Bisley, UK) to stimulate T cell proliferation. MNC cultured +/−PHA in the absence of MSC were used as controls for proliferation. For allostimulation assays, streptavidin-coated magnetic beads were used to produce CD4+ responder T cells (depletion of non-CD4 cells using a cocktail of biotinylated antibodies (CD8, -14, -15, -16, -19, -56, and HLA-DR)), and APC stimulators (depletion of lymphoid cells with anti-CD2, -3). The CD4+ preparations (>90% purity) were labelled with CFSE and mixed 1:1 with APC (50 k each). For accessory cell assays, PHA was added to the CD4:APC cultures at 0.5 ug/ml.

After 3-4 (PHA or bead stimulation) or 5-6 days (allostimulation) the cultures were analysed by flow cytofluorometry after staining for CD3 and CD4, and the CFSE dye-dilution profiles of CD3+CD4+ (CD4) and CD3+CD4- (CD8) lymphocytes were used to calculate proliferation indices (Lyons (2000)). To normalise the level of proliferation between experiments, the proliferation indices were calculated as: (proliferation index for condition X−proliferation index for unstimulated cells)/(proliferation index for PHA or bead stimulated control cells−proliferation index for unstimulated cells).

Drug Treatment and Suppression Assay Conditions.

Rapamycin was purchased as a 2.5 mg/ml DMSO solution, and Cyclosporin A, Everolimus and FK-506 monohydrate (all from Sigma) and Torin 1 (Tocris Bioscience, Bristol, UK) were dissolved in DMSO, with aliquots stored at −20° until use

Cells were cultured in T25 flasks with 5 mL standard growth medium until near confluence. Drug stock solutions were diluted in DMSO such that they were added to the cultures at <10 uL. These volumes of DMSO were shown to have no effect on MSC function in control experiments (data not shown). At times indicated in the text, the medium was removed from the cells, which were then rinsed with 5 mL calcium, magnesium-free phosphate-buffered saline (PBS). Trypsin was added in a 1 mL final volume until cells detached, then neutralised with 250 uL FCS and diluted with 5 mL PBS before centrifugation. The cell pellet was resuspended in growth medium at an initial concentration of 2.5×10⁵ cells/mL, with two further 5-fold dilutions made in medium in order to distribute 1,000; 5,000; 25,000 cells/well in 100 uL aliquots to U-bottom 96 well plates (BD Falcon, Oxford, UK). Control 96 well plates were made up with 100 uL/well growth medium alone. After 2-4 hours, CFSE-labelled responder cells (MNC or CD4+ T cells) were resuspended at 5×10⁵ cells/mL in growth medium and 100 uL aliquots distributed to the MSC and control plates together with the T cell stimulator as indicated in the text.

The sheep anti-Rapamycin Ig preparation and pre-immune serum were purchased from Aalto Bio Reagents (Dublin, Ireland).

### REFERENCES

Bernardo M E and Fibbe W E. ‘Safety and efficacy of mesenchymal stromal cell therapy in autoimmune disorders’ (2012) Annals of the New York Academy of Sciences 1266:107-117.

Borders E B, Bivona C, Medina P J. ‘Mammalian Target of Rapamycin: Biological Function and Target for Novel Anticancer Agents’ (2010) American Journal of Health-System Pharmacy 67:2095-2106.

Girdlestone J, Limbani V A, Cutler A J, Navarrete C V. ‘Efficient expansion of mesenchymal stromal cells from umbilical cord under low serum conditions’ (2009) Cytotherapy 11:738-48.

Lyons A B. ‘Analysing cell division in vivo and in vitro using flow cytometric measurement of CFSE dye dilution.’ (2000) J. Imm. Meth. 243: 147-154.

Thomson A W, Turnquist H R and Raimondi G. ‘Immunoregulatory functions of mTOR inhibition’ Nature Reviews Immunology (2009) 9:325

Thoreen C C, Kang S A, Chang J W, Liu Q, Zhang J, Gao Y, Reichling L J, Sim T, Sabatini D M, Gray N S. ‘An ATP-competitive mammalian target of rapamycin inhibitor reveals rapamycin-resistant functions of mTORC1.’ (2009) J. Biol. Chem. 284:8023-32

Yáñez J A, Forrest M L, Ohgami Y, Kwon G S, and Davies N M. ‘Pharmacometrics and delivery of novel nanoformulated PEG-b-poly(ε-caprolactone) micelles of rapamycin’. (2008) Cancer Chemother Pharmacol. 61: 133-144.

1. A method for preparing mesenchymal stem cells (MSCs) with enhanced immunosuppressive potency comprising the steps of:
(i) obtaining MSCs;
(ii) culturing and incubating the MSCs with an immunosuppressive agent; and
(iii) harvesting the MSCs.
thereby preparing MSCs with enhanced immunosuppressive potency.

2. The method according to claim 1, wherein the immunosuppressive agent is selected from rapamycin, Everolimus, Tacrolimus or cyclosporin A.

3. The method according to claim 2, wherein the immunosuppressive agent is rapamycin.

4. The method according to claim 1, wherein the cells in step (ii) are treated for less than 24 hours.

5. The method according to claim 4, wherein the cells are treated for less than 1 hour.

6. The method according to claim 1, wherein the immunosuppressive potency is increased potency to inhibit proliferation of CD4 and CD8 T lymphocytes.

7. An isolated MSC produced by the method according to claim 1.

8. The isolated MSC according to claim 7, which has an increased potency to inhibit proliferation of CD4 and CD8 T lymphocytes compared to an untreated MSC.

9. A pharmaceutical composition comprising the isolated MSC according to claim 7.

10. The pharmaceutical composition according to claim 9, which further comprises a pharmaceutically acceptable carrier, diluent, or excipient.

11. A method of suppressing an immune response in a subject, the method comprising administering to the subject the pharmaceutical composition according to claim 9.

12. A method for treating and/or preventing ‘Graft versus Host Disease’ (GvHD) or autoimmune disease in a subject, the method comprising administering to the subject the pharmaceutical composition according to claim 9.

13. A method for increasing the immunosuppressive potency of a cell, the method comprising:
(i) obtaining a cell;
(ii) culturing and incubating the cell with an immunosuppressive agent; and
(iii) harvesting the cell;
thereby increasing the immunosuppressive potency of the cell.

14. The method according to claim 13, wherein the cell is a primary fibroblast, a cell from a fibroblast cell line, an endothelial cell, or a haematopoietic cell.
